(19) United States
(12) Patent Application Publication
(10) Pub. No.: US 2025/0173872 A1
(43) Pub. Date: May 29, 2025

(54) INFORMATION PROCESSING APPARATUS, INFORMATION PROCESSING METHOD, AND STORAGE MEDIUM

(71) Applicants: Canon Kabushiki Kaisha, Tokyo (JP); CANON MEDICAL SYSTEMS CORPORATION, Tochigi (JP)

(72) Inventors: Masahiro YAKAMI, Kyoto-shi (JP); Ryo SAKAMOTO, Kyoto-shi (JP); Koji FUJIMOTO, Kyoto-shi (JP); Yutaka EMOTO, Kyoto-shi (JP); Takeshi KUBO, Kyoto-shi (JP); Shunjiro NOGUCHI, Kyoto-shi (JP); Toru KIKUCHI, Hino-shi (JP)

(73) Assignees: Canon Kabushiki Kaisha, Tokyo (JP); CANON MEDICAL SYSTEMS CORPORATION, Tochigi (JP)

(21) Appl. No.: 18/962,481

(22) Filed: Nov. 27, 2024

(30) Foreign Application Priority Data
Nov. 27, 2023 (JP) 2023-199862

(51) Int. Cl. G06T 7/00; G06T 3/40; G06T 7/70; G06V 10/74; G06V 10/764; G06V 10/82; G16H 50/20

(52) U.S. Cl. CPC G06T 7/0016; G06T 3/40; G06T 7/70; G06V 10/761; G16H 50/20; G06T 2207/20081; G06T 2207/20084; G06T 2207/30096; G06V 10/764; G06V 10/82; G06V 2201/03

(57) ABSTRACT

An information processing apparatus according to an embodiment includes processing circuitry configured to: acquire plural lesion candidates from a medical image; determine a group of lesion candidates from the plural lesion candidates acquired; select a representative lesion candidate from the lesion candidates forming the group; and display information related to the representative lesion candidate, together with and distinguishably from information related to lesion candidates other than the representative lesion candidate, the lesion candidates forming the same group as the representative lesion candidate.

### CROSS-REFERENCE TO RELATED APPLICATIONS

This application is based upon and claims the benefit of priority from Japanese Patent Application No. 2023-199862, filed on Nov. 27, 2023; the entire contents of which are incorporated herein by reference.

### FIELD

Embodiments described herein relate generally to an information processing apparatus, an information processing method, and a storage medium.

### BACKGROUND

Computer-Aided Detection (CADe), which is for analyzing a medical image by means of a calculator and detecting candidates for a lesion that is an abnormality associated with a disease, has been known. With the recent development of artificial intelligence (AI) technology, precision of detection of lesions targeted is improving. For example, a large number of lesions are detected in a single medical image for multiple lesions, such as multiple lung metastases (metastatic pulmonary tumors), polycystic kidney, and multiple hepatic tumors.

A medical doctor needs to make a diagnosis by checking individual images of a large number of such lesions detected and determining whether the lesions are lesions associated with a multifocal disease or lesions associated with different diseases.

### BRIEF DESCRIPTION OF THE DRAWINGS

FIG. 1 is a diagram illustrating a configuration of an information processing system according to a first embodiment and a second embodiment;

FIG. 2 is a diagram illustrating a hardware configuration of an information processing apparatus according to the first embodiment and the second embodiment;

FIG. 3 is a diagram illustrating a functional configuration of functions of the information processing apparatus according to the first embodiment and the second embodiment;

FIG. 4 is a diagram illustrating an example of a user interface screen on the information processing apparatus according to the first embodiment and the second embodiment;

FIG. 5 is a diagram illustrating an example of a user interface screen upon a representative lesion being specified at the information processing apparatus according to the first embodiment and the second embodiment;

FIG. 6 is a flowchart illustrating an image display process by the information processing apparatus according to the first embodiment and the second embodiment; and

FIG. 7 is a diagram illustrating an example of a user interface screen on an information processing apparatus according to the second embodiment.

### DETAILED DESCRIPTION

An information processing apparatus according to an embodiment includes processing circuitry configured to acquire plural lesion candidates from a medical image, determine a group of lesion candidates from the plural lesion candidates acquired, select a representative lesion candidate from the lesion candidates forming the group, and display information related to the representative lesion candidate, together with and distinguishably from information related to lesion candidates forming the same group as the representative lesion candidate, the lesion candidates being other than the representative lesion candidate.

Furthermore, an information processing method according to an embodiment includes: acquiring plural lesion candidates from a medical image; determining a group of lesion candidates from the plural lesion candidates acquired; selecting a representative lesion candidate from the lesion candidates forming the group; and displaying information related to the representative lesion candidate, together with and distinguishably from information related to lesion candidates forming the same group as the representative lesion candidate, the lesion candidates being other than the representative lesion candidate.

Furthermore, a computer-readable non-transitory recording medium according to an embodiment has a program recorded therein, the program causing a computer to execute processes of: acquiring plural lesion candidates from a medical image; determining a group of lesion candidates from the plural lesion candidates acquired; selecting a representative lesion candidate from the lesion candidates forming the group; and displaying information related to the representative lesion candidate, together with and distinguishably from information related to lesion candidates forming the same group as the representative lesion candidate, the lesion candidates being other than the representative lesion candidate.

A preferred embodiment of the present invention will hereinafter be described in detail by reference to the appended drawings. The same number will be assigned to any item that will be described with respect to other embodiments and description thereof will be omitted, unless otherwise stated particularly. Configurations of the embodiments described hereinafter are just examples and the present invention is not to be limited to the configurations illustrated in the drawings.

### First Embodiment

An information processing apparatus that displays a medical image, such as an X-ray computed tomography (CT) image or a magnetic resonance imaging (MRI) image, will be described with respect to a first embodiment. A medical doctor displays a medical image on the information processing apparatus, makes a diagnosis, and generates a result of the diagnosis as an image diagnosis report (hereinafter, referred to as the “image interpretation report” or simply as the “report”) in a report system not illustrated in the drawings.

At the information processing apparatus according to the embodiment, one or more CADes operate, detect candidates (hereinafter referred to as “lesion candidates” or simply as “lesions”) for a lesion that is a clinical abnormality appearing on the medical image, and present information indicating that the lesions have been detected, to a user. The user specifies one or more lesions of the lesions presented and makes a diagnosis by observing an image or images of the one or more lesions. A case where the medical image is an X-ray CT image and lesions to be detected by CADe are pulmonary nodules will be described with respect to this embodiment, but without being limited to this case, the medical image may be any other medical image, such as an MRI image, an ultrasound image, a mammographic image, or a simple X-ray image, and the lesions may be any other lesions, such as hepatophyma, renal mass, encephaloma, or osseous lesions.

### System Configuration

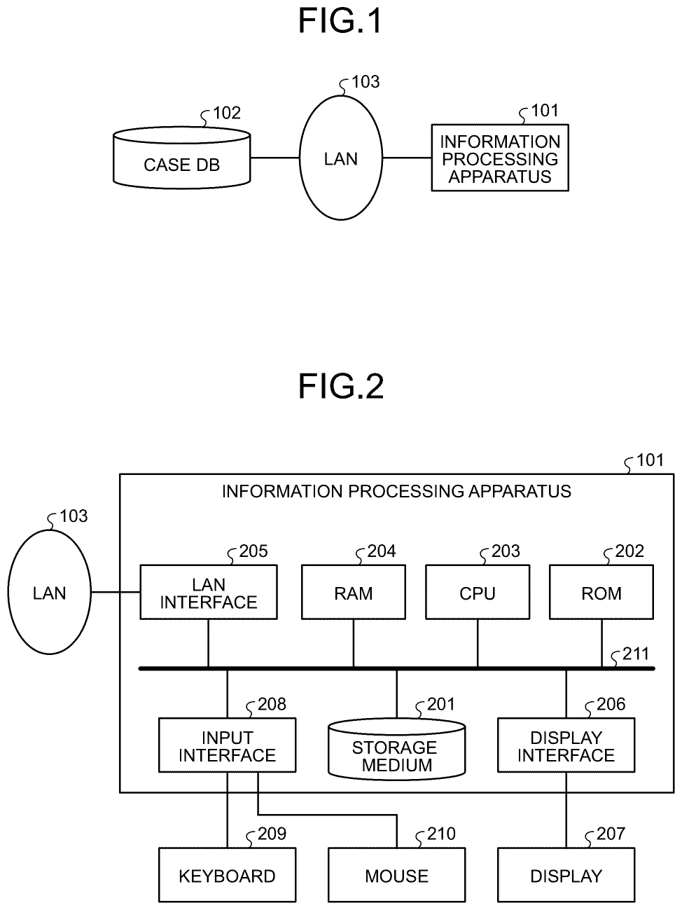

FIG. 1 is a diagram illustrating a configuration of an information processing system according to this embodiment.

In FIG. 1, the information processing system includes a case database (hereinafter, referred to as the “case DB”) 102, an information processing apparatus 101, and a local area network (LAN) 103.

The information processing apparatus 101 acquires and displays medical image data (hereinafter, also referred to as the “medical image”) from the case DB 102. Furthermore, the information processing apparatus 101 performs detection of any lesion from the medical image and displays a result of the detection. The information processing apparatus 101 also automatically generates and displays an example of a sentence to be written in an image interpretation report (hereinafter, referred to as the “image interpretation report” or “report”) for any lesion detected.

The case DB 102 stores therein medical images captured by an apparatus that captures medical images, such as a CT apparatus (not illustrated in the drawings). Furthermore, the case DB 102 provides a medical image to the information processing apparatus 101 via the LAN 103. Specifically, the case DB 102 according to the embodiment is a known picture archiving and communication system (PACS).

### Hardware Configuration

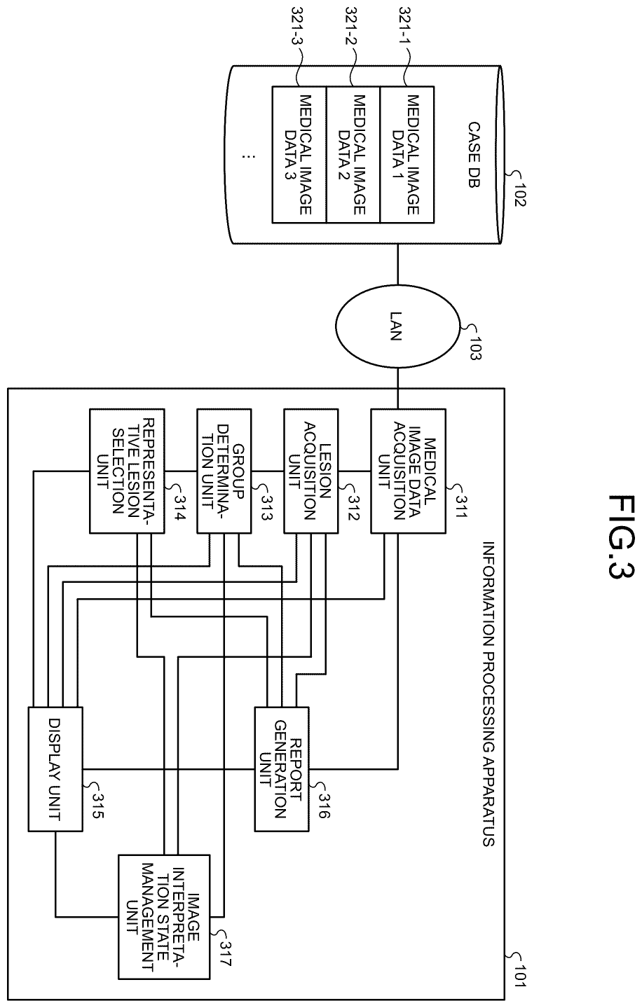

FIG. 2 is a diagram illustrating a hardware configuration of the information processing apparatus according to the embodiment.

In FIG. 2, the information processing apparatus 101 includes a storage medium 201, a read only memory (ROM) 202, a central processing unit (CPU) 203, and a random access memory (RAM) 204. The information processing apparatus 101 also includes a LAN interface 205, an input interface 208, a display interface 206, and an internal bus 211.

The storage medium 201 is a storage medium, such as a solid state drive (SSD), that stores an operating system (OS), processing programs for executing various kinds of processing according to the embodiment, and various kinds of information. The ROM 202 stores a program, such as a basic input output system (BIOS), for initializing hardware and booting the OS. The CPU 203 performs arithmetic processing in executing the BIOS, the OS, and the processing programs. The RAM 204 temporarily stores information for the CPU 203 to execute the BIOS, OS, and programs. The LAN interface 205 is an interface compatible with a standard, such as the Institute of Electrical and Electronics Engineers (IEEE) 802.3ab, and for implementing communication via the LAN 103. A reference numeral 207 corresponds to a display, such as a liquid crystal display (LCD), to display a user interface screen. The display interface 206 converts screen information to be displayed on the display 207 to a signal for display control and outputs the signal to the display 207. A reference numeral 209 corresponds to a keyboard to implement key input and a reference numeral 210 corresponds to a mouse to implement specification of a coordinate position on a display screen and input of button operation. The input interface 208 receives signals based on a depression of a key, a button click, and a movement of coordinates, from the keyboard 209 or the mouse 210. The internal bus 211 implements transmission of signals in communication between blocks.

### Functional Configuration

FIG. 3 is a diagram illustrating a functional configuration of the information processing apparatus 101 according to the embodiment.

In FIG. 3, the information processing apparatus 101 includes a medical image data acquisition unit 311, a lesion acquisition unit 312, a group determination unit 313, a representative lesion selection unit 314, a display unit 315, a report generation unit 316, and an image interpretation state management unit 317. The case DB 102 stores medical image data 321-i (i=1, 2, 3, . . . ). The medical image data 321-i (i=1, 2, 3, . . . ) are a file conforming to the international standard, Digital Imaging and Communications in Medicine (DICOM).

For example, the medical image data acquisition unit 311, the lesion acquisition unit 312, the group determination unit 313, the representative lesion selection unit 314, the display unit 315, the report generation unit 316, and the image interpretation state management unit 317 are implemented by processing circuitry, such as the CPU 203, executing corresponding programs. For example, by reading and executing a program corresponding to the medical image data acquisition unit 311 from the storage medium 201, the processing circuitry functions as the medical image data acquisition unit 311. Similarly, the processing circuitry is capable of functioning as the lesion acquisition unit 312, the group determination unit 313, the representative lesion selection unit 314, the display unit 315, the report generation unit 316, and the image interpretation state management unit 317.

In the information processing apparatus 101 illustrated in FIG. 2 and FIG. 3, each processing function has been stored in the storage medium 201 in the form of a program executable by a computer. The processing circuitry is a processor that implements the functions corresponding to the programs by reading and executing the programs from the storage medium 201. In other words, the processing circuitry in a state of having read the programs has the functions corresponding to the read programs.

It has been described with respect to FIG. 2 and FIG. 3 that the processing functions, such as the medical image data acquisition unit 311, the lesion acquisition unit 312, the group determination unit 313, the representative lesion selection unit 314, the display unit 315, the report generation unit 316, and the image interpretation state management unit 317, are implemented by the single CPU 203, but the embodiment is not to be limited to this example. For example, the processing circuitry may be formed of a combination of plural independent processors and the processing functions may be implemented by these processors respectively executing the programs. Furthermore, the processing functions may be implemented by being distributed to plural pieces of processing circuitry or integrated into a single piece of processing circuitry, as appropriate. Furthermore, an example where the programs corresponding to the processing functions are stored in the single storage medium 201 has been described with respect to FIG. 2, but the programs corresponding to the processing functions may be stored in plural storage media 201 in a distributed manner and the programs may be respectively read and executed from the storage media 201.

Furthermore, the processing circuitry may implement a function by using a processor of an external device connected via a network. For example, by reading and executing the programs corresponding to the respective functions from the storage medium 201 and using, as a computational resource, a server group (a cloud) connected to the information processing apparatus 101 via the network, the processing circuitry may implement the processing functions illustrated in FIG. 3.

The term, “processor”, used in the above description means, for example: a CPU; a graphics processing unit (GPU); or a circuit, such as an application specific integrated circuit (ASIC) or a programmable logic device (for example, a simple programmable logic device (SPLD), a complex programmable logic device (CPLD), or a field programmable gate array (FPGA)). That is, the processing functions according to the embodiment may be implemented by the CPU 203 illustrated in FIG. 2 or may be implemented by another processor. In a case where the processor is the CPU 203, the processor implements the functions by reading and executing the programs stored in the storage medium 201. In contrast, in a case where the processor is, for example, an ASIC, instead of the programs being stored in the storage medium 201, the functions are directly incorporated, as logic circuits, in circuitry of the processor. Each processor according to the embodiment is not necessarily configured as a single piece of circuitry and plural independent pieces of circuitry may be combined together to be configured as a single processor for their functions to be implemented. Furthermore, plural components in each drawing may be integrated into a single processor for their functions to be implemented.

The medical image data acquisition unit 311 acquires the medical image data 321-i (i=1, 2, 3, . . . ) to be subjected to image interpretation, from the case DB 102 via the LAN 103. This acquisition of the medical image data 321-i (i=1, 2, 3, . . . ) conforms to DICOM. The medical image data to be subjected to image interpretation are assumed to have been specified by a user beforehand.

The lesion acquisition unit 312 acquires plural lesion candidates from a medical image. For example, the lesion acquisition unit 312 detects any lesion from the medical image data 321-i (i=1, 2, 3, . . . ) acquired by the medical image data acquisition unit 311. The lesion acquisition unit 312 is an example of an acquisition means.

In this embodiment, pulmonary nodules are detected from an X-ray CT image having a chest captured therein. Information related to a result of lesion detection (hereinafter, referred to as the “lesion detection result”) includes information indicating areas of individual lesions detected (hereinafter, referred to as “lesion areas”) and information indicating classifications of lesions (hereinafter, referred to as “lesion classifications”). In this embodiment, a lesion area includes coordinate information on the center of a lesion (hereinafter, referred to as the “lesion position”) and information related to a size of the lesion (hereinafter, referred to as the “lesion size”). For example, in a case where two lesions have been detected, a lesion detection result obtained is a sequence of three-element tuples each including a lesion classification, a lesion position, and a lesion size, “[(LT1, (X1, Y1, Z1), SZ1), (LT2, (X2, Y2, Z2), SZ2)]”. In this case, LTn (n=1, 2) is the lesion classification, Xn (n=1, 2) is the coordinate value of the lesion position on a horizontal axis, Yn (n=1, 2) is the coordinate value of the lesion position on a vertical axis, Zn (n=1, 2) is the coordinate value of the lesion position in a depth direction, and SZn (n=1, 2) is the lesion size. Furthermore, to any lesion that has been detected, an identifier (hereinafter, referred to as the “lesion ID”) to uniquely identify the lesion is assigned. For example, a lesion ID, “L1”, is assigned to the lesion corresponding to “(LT1, (X1, Y1, Z1), SZ1)”, and a lesion ID, “L2”, is assigned to the lesion corresponding to “(LT2, (X2, Y2, Z2), SZ2)”. A lesion area may include any other information indicating the area, such as coordinate information on a bounding box circumscribing the lesion, coordinate information on each pixel corresponding to the lesion area, or mask image data having the lesion area labelled therein (hereinafter, “mask image data” may simply be referred to as the “mask image”).

A detector obtained by machine training of a convolutional neural network (CNN) is used in detection of lesions. In the machine training of the CNN, plural medical images are collected beforehand and mask images having lesion areas labelled therein are generated beforehand respectively for the medical images collected. The machine training is then performed with training data that are pairs of the medical images and the mask images. Specifically, a medical image is input to the CNN, parameters of the CNN are changed so that a difference between a mask image output from the CNN and a mask image in the training data is decreased, and this process is repeated. Examples of the parameters of the CNN include kernels of convolutional operations, and weight and bias values of a fully connected layer. The lesion acquisition unit 312 may use a deep neural network (DNN) instead of a CNN. Furthermore, a non-deep learning determiner may be used by: division of a medical image into areas each having a predetermined size; extraction of image features by a publicly known method, such as radiomics; and determination of presence of any lesion in each area using a support vector machine (SVM). Output of the detector or determiner is converted in the above described form of a lesion detection result as appropriate.

The lesion acquisition unit 312 may detect, for example, lesions of plural classifications, such as pulmonary nodules and hepatophyma. In a case where lesions of plural classifications are to be detected, detectors or determiners may be included for the respective lesion classifications or the lesions of plural classifications may be detected or determined by means of a single detector or determiner. In a case where lesions of plural classifications are to be detected or determined by means of a single detector or determiner, machine training is performed by use of training data that are multiple-valued mask images having different label values for the respective lesion classifications. The lesion acquisition unit 312 may acquire information on a result of detection processing executed at an external server apparatus (not illustrated in the drawings), for example, via the LAN 103.

Instead of obtaining a lesion detection result by image analysis using a CNN, the lesion acquisition unit 312 may acquire a lesion detection result on the basis of user operation on a user interface screen not illustrated in the drawings. Specifically, a user specifies a position and a size of a lesion in a medical image displayed. Furthermore, a lesion detection result may be a combination of a lesion detection result obtained by image analysis and a lesion detection result specified by a user.

The group determination unit 313 determines a group of lesion candidates from plural lesion candidates acquired by the lesion acquisition unit 312. The group determination unit 313 is an example of a determination means. For example, the group determination unit 313 acquires a lesion detection result from the lesion acquisition unit 312 and calculates degrees of similarity between lesions. Subsequently, the group determination unit 313 determines, as a group of similar lesions, a collection of plural lesions having degrees of mutual similarity higher than a predetermined value. That is, the group determination unit 313 acquires degrees of similarity between each of the plural lesion candidates acquired and the rest of the plural lesion candidates and determines a group by using the degrees of similarity.

Information related to a result of determining a group (hereinafter, referred to as the “similar lesion group”) is a sequence of lesion IDs included in the group. For example, in a case where the lesion IDs are L1, L2, and L3, a sequence, “[L1, L2, L3]”, is the similar lesion group and an identifier, for example, “LG1”, that uniquely identifies the group is assigned to the similar lesion group.

Furthermore, the group determination unit 313 may output information (hereinafter, referred to as the “degree of similarity”) related to a degree of similarity calculated in determining the group. For example, a degree of similarity has a real value ranging from 0.0 to 1.0. For example, in a case where the degree of similarity between the lesion L1 and the lesion L2 is 0.79, information related to the degree of similarity is a three-element tuple, “(L1, L2, 0.79)”. Such degrees of similarity may be output for all of combinations of lesions detected or such a degree of similarity may be generated for only a combination of lesions forming one group.

For example, the group determination unit 313 vectorizes image features on images corresponding to lesion areas and calculates degrees of similarity from distances therebetween. The degrees of similarity are normalized such that those with larger distances have degrees of similarity closer to 0.0 and those with smaller distances have degrees of similarity closer to 1.0. Furthermore, publicly known radiomics features may be used, for example, as the image features. The image features may be features based on histograms, such as histograms of oriented gradients (HOG) or scaled invariance feature transform (SIFT). The image features may be features, such as the major axis (lesion size) and minor axis of the lesion area, the major axis, minor axis, and volume for every third cross section, and the area and luminance distribution for every third cross section. Furthermore, the image features may be features based on an autoencoder using a CNN or an image filter, such as a Laplacian of Gaussian (LOG) filter. Furthermore, inferences of differential diagnoses and image findings, such as margin characteristics, overall shapes, and internal concentrations, may be made by means of a CNN or image analysis and results of the inferences may be used, instead of the image features. In a case where image findings are inferred by means of a CNN, machine training is performed so that image findings are output in response to input of an image of a lesion area, with training data that are images of lesion areas and correct answer values for image findings. For differential diagnoses, machine learning is similarly performed so that a differential diagnosis, for example, any one of “primary lung cancer”, “metastatic lung cancer”, and “benign tubercle”, is output in response to input of an image of a lesion area. Correct answer values for image findings are, for example, “smooth”, “partly irregular”, and “irregular” for margin characteristics, and “spherical”, “lobulated”, “polyhedral”, and “irregular” for overall shapes. An internal concentration is determined on the basis of a ratio between concentration (HU value) ranges corresponding a solid component and a ground glass component by histogram analysis of a lesion area and is classified as “solid”, “part-solid”, or “pure-GGN”, these classifications being listed herein in descending order of the concentration of the solid component. In determination of a similar lesion group, a classifier, such as a CNN, a SVM, or a DNN, which has been subjected to machine training with a binary classification problem for “being similar” or “not being similar” may be used. A degree of similarity in this case is a likelihood of being similar.

The representative lesion selection unit 314 selects a representative lesion candidate (hereinafter, referred to as the “representative lesion”) from lesion candidates forming a similar lesion group determined by the group determination unit 313. The representative lesion selection unit 314 is an example of a selection means. Furthermore, the representative lesion selection unit 314 outputs information related to the representative lesion.

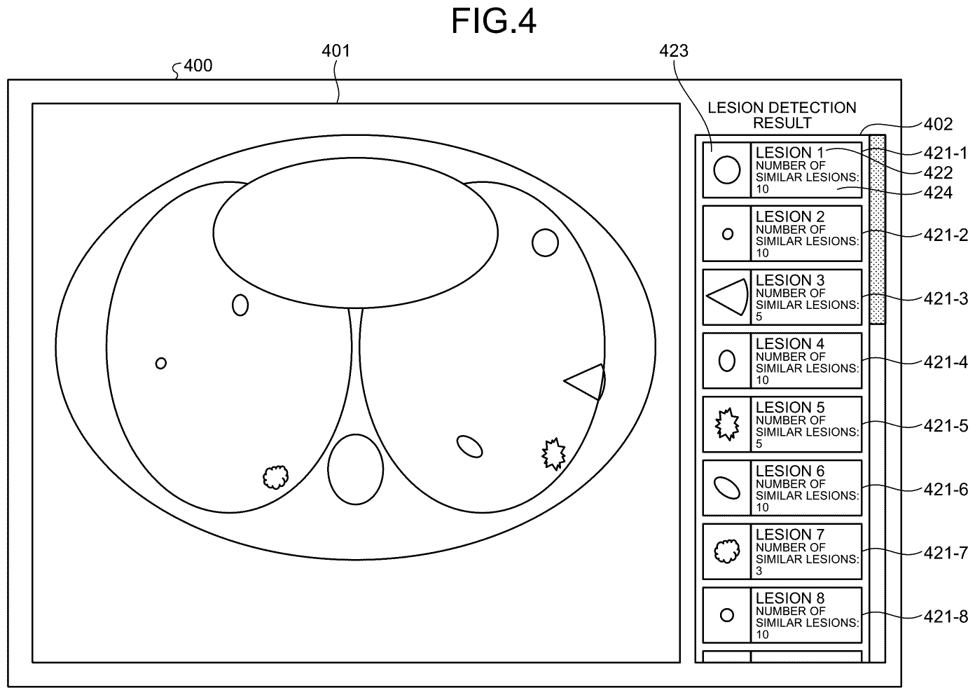

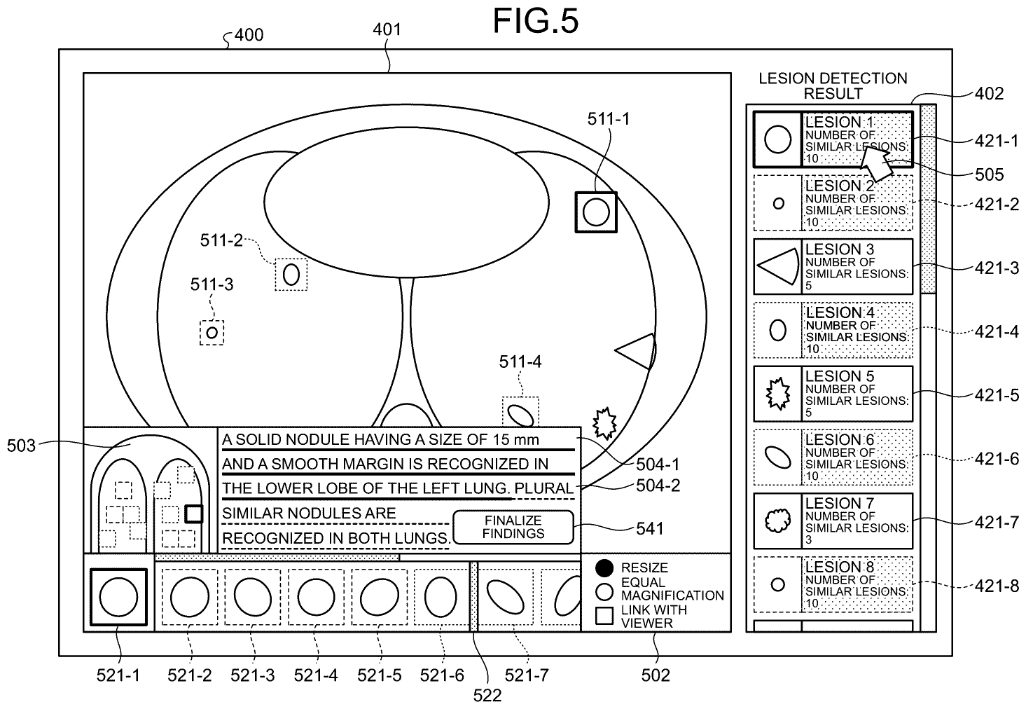

For example, the representative lesion selection unit 314 selects and outputs the lesion L2 as a lesion representing the similar lesion group LG1. In this embodiment, the representative lesion selection unit 314 selects a lesion specified by user operation, as a representative lesion of a similar lesion group that the lesion belongs to. That is, the representative lesion selection unit 314 selects a representative lesion in response to reception of user operation. The specification by the user operation is performed, for example, via a user interface screen described by use of FIG. 4 and FIG. 5.

In response to a representative lesion being specified, the display unit 315 displays information related to the representative lesion specified, together with information related to lesion candidates (hereinafter, referred to as “similar lesions”) other than the representative lesion, the lesion candidates forming the same group as the representative lesion. Information related to a lesion is, for example, a partial image, a lesion position, and an image interpretation report that correspond to the lesion area in the medical image. Furthermore, the display unit 315 displays the information related to the representative lesion and the information related to the similar lesions distinguishably from each other. That is, the display unit 315 displays the information related to the representative lesion, together with and distinguishably from the information related to the similar lesions. The display unit 315 is an example of a display means.

The information may be made distinguishable by changing, for example, the thicknesses, line types, and/or line colors of frame lines for display of the images and information. For text, the information may be made distinguishable by changing, for example: the thicknesses, line types, and/or line colors of underlines; the character colors; and/or the background colors. Furthermore, in displaying the identifier of the representative lesion, the display unit 315 may display the number (count) of the similar lesions for that representative lesion. An image interpretation report related to a lesion is generated by the report generation unit 316 described later.

In response to a representative lesion being specified, the report generation unit 316 generates an image interpretation report related to the representative lesion specified. Furthermore, the report generation unit 316 generates not only the report related to the presentative lesion but also a report related to the similar lesions. That is, the report generation unit 316 performs the same kind of processing also for the similar lesions, according to processing for the representative lesion. In other words, the report generation unit 316 performs the same kind of processing for the representative lesion and the similar lesions. The report generation unit 316 is an example of a processing means.

Generation of a report related to a representative lesion is implemented by, for example, an inference of image findings from an image of the lesion area and application of the inferred image findings to a template. The template is, for example, “{Lesion name according to concentration} having a size of {lesion size} mm and a {margin characteristic} margin is recognized in the {region}.” In this template, “{ }” is a part substituted by an image finding. Specifically, if the region is “the lower lobe of the left lung”, the lesion size is “15 (mm)”, the margin characteristic is “smooth”, and the concentration is “solid” (the lesion name according to concentration is “solid nodule”), the report generated is “A solid nodule having a size of 15 mm and a smooth margin is recognized in the lower lobe of the left lung.”. A lesion name according to concentration is a name (text information) of a lesion uniquely associated with a concentration. Furthermore, for a report related to similar lesions, a distribution is evaluated from lesion positions of the similar lesions and the report would read, “Plural similar nodules are recognized in {distribution}.”. In a case where the distribution evaluated is for “both lungs”, the report would read, “Plural similar nodules are recognized in both lungs”. Examples of other distributions may be for “the same lung” and “the same lobe”. In determination of a region, a result of organ segmentation related to pulmonary lobes and not illustrated in the drawings and lesion positions are used. These reports may be generated by use of a deep learning model, such as a recurrent neural network (RNN) or a long short-term memory (LSTM). These models are subjected to machine training using training data having input that is rows of image findings and output that is report sentences.

The image interpretation state management unit 317 performs management of a state (hereinafter, simply referred to as the “image interpretation state) related to image interpretation of a lesion detected. An image interpretation state is information indicating whether or not image interpretation of a lesion has been completed. The image interpretation having been completed is a state where a report related to the lesion has been finalized. The image interpretation state management unit 317 may change a state related to image interpretation of similar lesions, according to a change made to a state related to image interpretation of a representative lesion. Specifically, in response to finalization of reports on a representative lesion and similar lesions by user operation, the reports having been generated by the report generation unit 316, the image interpretation state management unit 317 transfers the reports to the report system not illustrated in the drawings and changes the image interpretation state of the representative lesion to “completed”. The image interpretation state management unit 317 also changes the image interpretation state of the similar lesions to “completed”. That is, the image interpretation state management unit 317 performs the same kind of processing also for the similar lesions, according to processing for the representative lesion. In other words, the image interpretation state management unit 317 performs the same kind of processing for the representative lesion and the similar lesions. The image interpretation state management unit 317 is an example of a processing means. Information related to a lesion, for which image interpretation has been completed, is displayed distinguishably from a lesion “uncompleted” by being grayed out or the report being disabled to be changed.

### User Interface Screen

FIG. 4 is a diagram illustrating an example of a user interface screen on the information processing apparatus according to the embodiment. A user interface screen 400 is displayed on the display 207 by the display unit 315 and various kinds of operation by a user are input via the keyboard 209 and the mouse 210.

In FIG. 4, the user interface screen 400 includes a medical image display area 401 and a lesion detection result display area 402.

The medical image display area 401 has a medical image displayed therein, the medical image having been acquired by the medical image data acquisition unit 311. For the medical image display area 401, the window level/window width (WL/WW) of the image, the position of the cross section (hereinafter, also referred to as the “slice”), and the scale of enlargement, for example, are able to be changed according to operation by means of the keyboard 209 or the mouse 210.

The lesion detection result display area 402 has a list of lesion detection results 421-i (i=1, 2, 3, . . . ) displayed therein, the lesion detection results 421-i (i=1, 2, 3, . . . ) being related to lesions detected by the lesion acquisition unit 312. For a lesion detection result 421-i, text 422 identifying the lesion detected, a representative image 423 of the lesion detected, and the number of similar lesions 424 for the lesion detected are displayed. The representative image 423 of the lesion is an image cut out from the medical image on the basis of the lesion position of the lesion detected, the image corresponding to a predetermined range of the medical image and being, for example, an axial cross section having a predetermined size from the center of the lesion area. In a case where the lesion size is larger than the display area of the representative image, a predetermined range based on the lesion size is cut out and displayed in a reduced manner. In a case where plural classifications of lesions are able to be detected, information related to the classifications of the lesions detected is also displayed in the lesion detection results 421-i. The representative image may be an image of any other cross section, such as an image of a cross section where the lesion has the largest major axis in the axial cross section. In a case where the lesion size is smaller than a predetermined size, the representative image may be displayed in an enlarged manner.

FIG. 5 is a diagram illustrating an example of a user interface screen upon specification of a representative lesion by the information processing apparatus according to the embodiment. Various kinds of display illustrated in FIG. 5 are also displayed on the display 207 by the display unit 315. Furthermore, various kinds of operation by a user are input via the keyboard 209 and the mouse 210.

In FIG. 5, a reference numeral 505 corresponds to a mouse pointer and a lesion corresponding to a lesion detection result 421-1 has been specified by operation, such as a mouse click by a user. In a case described with respect to this embodiment, a lesion specified by a user is selected as a representative lesion of a similar lesion group that the lesion belongs to. In response to detection of specification of a representative lesion, the display unit 315 highlights the lesion detection result 421-1 as information related to the representative lesion specified. Furthermore, together with the lesion detection result for the representative lesion, the display unit 315 highlights lesion detection results for similar lesions for the representative lesion. Specifically, the display unit 315 highlights lesion detection results 421-2, 421-4, 421-6, and 421-8. Furthermore, the display unit 315 displays the lesion detection result for the representative lesion and the lesion detection results for the similar lesions for the representative lesion, distinguishably from each other. Specifically, the display unit 315 displays a frame of the lesion detection result 421-1 of the representative lesion with a bold solid line and frames of the lesion detection results 421-2, 421-4, 421-6, and 421-8 for the similar lesions with a thin broken line or dotted line. Furthermore, the display unit 315 changes the display mode of the lesion detection results for the similar lesions, on the basis of degrees of similarity between the representative lesion and the individual similar lesions. Specifically, the display unit 315 displays the frame of any lesion detection result for a lesion having a degree of similarity higher than a predetermined value with a broken line and the frame of any lesion detection result for a lesion having a degree of similarity lower than the predetermined value with a dotted line.

Furthermore, in response to detection of specification of a representative lesion, the display unit 315 changes the slice position of an image to be displayed in the medical image display area 401 to the center position of the lesion area of the representative lesion specified. The display unit 315 then displays, as information related to the representative lesion, a region of interest 511-1 (a rectangle surrounding the lesion) for the representative lesion, on the displayed image. Together with the region of interest for the representative lesion, the display unit 315 displays regions of interest for lesions included in the image being displayed, the lesions being some of the similar lesions for the representative lesion. Specifically, the display unit 315 displays regions of interest 511-2, 511-3, and 511-4 for the similar lesions. Furthermore, the display unit 315 displays the region of interest for the representative lesion and the regions of interest for the similar lesions for the representative lesion, distinguishably from each other. Specifically, the display unit 315 displays a frame of the region of interest 511-1 of the representative lesion with a bold solid line and frames of the regions of interest 511-2, 511-3, and 511-4 for the similar lesions with a thin broken line or dotted line. Similarly to the case for the lesion detection results, the display unit 315 changes the display mode of the regions of interest for the similar lesions, on the basis of the degrees of similarity between the representative lesion and the individual similar lesions. Specifically, the display unit 315 displays any lesion having a degree of similarity higher than a predetermined value with a broken line and any lesion having a degree of similarity lower than the predetermined value with a dotted line.

Furthermore, in response to detection of specification of a representative lesion, the display unit 315 displays, as information related to the representative lesion, a partial image 521-1 that is an image corresponding to a lesion area of the representative lesion in the medical image.

Furthermore, together with the partial image for the representative lesion, the display unit 315 displays partial images for similar lesions for the representative lesion. Specifically, the display unit 315 displays partial images 521-i (i=2, . . . , 6). Furthermore, the display unit 315 displays the partial image for the representative lesion and the partial images for the similar lesions for the representative lesion, distinguishably from each other. Specifically, the display unit 315 displays a frame of the partial image 521-1 for the representative lesion with a bold solid line and frames of the partial images 521-i (i=2, . . . , 6) with a thin broken line or dotted line. Similarly to the case for the lesion detection results, the display unit 315 changes the display mode of the partial images for the similar lesions, on the basis of the degrees of similarity between the representative lesion and the individual similar lesions. Specifically, the display unit 315 displays any lesion having a degree of similarity higher than a predetermined value with a broken line and any lesion having a degree of similarity lower than the predetermined value with a dotted line. Furthermore, the display unit 315 arranges the partial images for the similar lesions on the screen in order based on the degrees of similarity. Specifically, the display unit 315 arranges the partial images 521-i (i=2, . . . , 6) for the similar lesions in descending order of the degrees of similarity, on the screen. On the basis of the degree of similarity corresponding to a position specified by user operation on displayed information related to the similar lesions, the group determination unit 313 is able to determine a group. For example, the group determination unit 313 is able to acquire the degree of similarity of a lesion to be excluded from a similar lesion group, on the basis of specification by a user. Specifically, in response to a user specifying a boundary between partial images, for example, a boundary 522 between the partial image 521-6 and the partial image 521-7, the group determination unit 313 excludes any lesion having a degree of similarity lower than that of the similar lesion corresponding to the partial image 521-7 from that similar lesion group. Furthermore, the group determination unit 313 is also able to receive, from a user, specification of a lesion itself to be excluded from a similar lesion group. For example, the user is able to exclude a lesion from a similar lesion group by placing a mouse pointer on a partial image and depressing a delete key. Furthermore, the display unit 315 is able to change the position of a slice to be displayed in a partial image, on the basis of user operation on the partial image. That is, the display unit 315 is able to change the position of a cross section to be displayed, in the partial image, by receiving operation on the partial image. For example, in response to detection of operation, such as rotation of a mouse wheel or depression of an up or down key on a partial image 521-i (i=1, . . . , 6), the display unit 315 changes the position of a slice to be displayed in the partial image. Furthermore, on the basis of user operation on a partial image, the display unit 315 is able to change the slice position of a medical image to be displayed in the medical image display area 401 to a slice position of a lesion corresponding to the partial image. Specifically, in response to detection of operation, such as a mouse click, on a partial image, the display unit 315 changes the slice position of the medical image to be displayed in the medical image display area 401 to a slice position corresponding to the partial image clicked on. Furthermore, in displaying a partial image, the display unit 315 is able to receive specification of whether to perform individual resizing (enlargement or reduction) on the basis of sizes of individual lesions, to have the same magnification for plural lesions, or to set the same magnification as the medical image displayed in the medical image display area 401. Specifically, “Resize” and “Equal magnification” are able to be specified by radio buttons in a partial image display control specification area 502. In response to detection of specification of “Resize”, the display unit 315 displays images of lesion areas in an enlarged or reduced manner such that the lesions are generally equal in size in the partial images. Furthermore, in response to detection of specification of “Equal magnification”, the display unit 315 displays the images of the lesion areas at a predetermined magnification for all of the partial images 521-i (i=1, . . . , 6). Furthermore, in response to detection of a check in a checkbox for “Link with viewer” in the partial image display control specification area 502, the display unit 315 displays the images of the lesion areas at a magnification that is the same as the magnification of the medical image displayed in the medical image display area 401, for all of the partial images 521-i (i=1, . . . , 6). In a case where the display unit 315 has detected that an image of a lesion area does not fit in a partial image 521-i (i=1, . . . , 6), the display unit 315 may reduce the image to a size that fits in the partial image 521-i. Furthermore, the display unit 315 may provide a limitation such that the magnification does not become equal to or larger than a predetermined magnification.

Furthermore, in response to detection of specification of a representative lesion, the display unit 315 displays a lesion position 503 on the basis of its lesion position. The lesion position 503 is a display having a figure (rectangle) indicating the position of the representative lesion specified and figures (rectangles) indicating the positions of the similar lesions, the figure and the figures having been arranged at corresponding positions on a two-dimensional schema. The figures, such as the rectangles, depicted in the lesion position 503 are an example of information related to a position. That is, the display unit 315 displays information related to the positions of the similar lesions, together with information related to the position of the representative lesion. Similarly to the other displays, the display unit 315 displays the position of the representative lesion and the positions of the similar lesions, distinguishably from each other. Specifically, the display unit 315 displays the representative lesion with a bold solid line and the similar lesions with a thin broken line or dotted line. Furthermore, the display unit 315 displays the positions of the similar lesions differently on the basis of the degrees of similarity between the representative lesion and the similar lesions. Specifically, the display unit 315 displays any lesion having a degree of similarity higher than a predetermined value with a broken line and any lesion having a degree of similarity lower than the predetermined value with a dotted line. In response to detection of specification of a figure indicating a position of a lesion by a user, the display unit 315 changes the slice position of the medical image to be displayed in the medical image display area 401 to a slice position corresponding to the position of the lesion specified.

Furthermore, in response to detection of specification of a representative lesion, the display unit 315 displays an image interpretation report 504-1 related to the representative lesion specified, together with an image interpretation report 504-2 on the similar lesions. Furthermore, the display unit 315 displays the image interpretation report on the representative lesion and the image interpretation report on the similar lesions, distinguishably from each other. Specifically, the display unit 315 displays an underline in the image interpretation report 504-1 on the representative lesion with a bold solid line and an underline in the image interpretation report 504-2 on the similar lesions with a thin broken line. The image interpretation reports 504-1 and 504-2 are generated by the report generation unit 316. The image interpretation report 504-1 is an example of findings for a representative lesion. The image interpretation report 504-2 is an example of findings for similar lesions. That is, the report generation unit 316 acquires findings for similar lesions according to acquisition of findings for a representative lesion and displays the findings for the similar lesions, together with the findings for the representative lesion. Text of the image interpretation reports 504-1 and 504-2 is able to be edited in any way by a user clicking on a position to be edited with a mouse and performing input using a keyboard.

Furthermore, the display unit 315 displays a finding finalization button 541, together with the image interpretation reports 504-1 and 504-2. In response to detection of a mouse click on the finding finalization button 541 by a user, the image interpretation reports 504-1 and 504-2 are transmitted to the report system not illustrated in the drawings, and the image interpretation state management unit 317 changes the image interpretation state of the corresponding representative lesion to “completed” and also changes the image interpretation state of the similar lesions to “completed”. The display unit 315 changes information related to any lesion having its image interpretation state changed to “completed” to a gray character string or frame line to display the information distinguishably from any lesion “uncompleted”.

### Process Flow

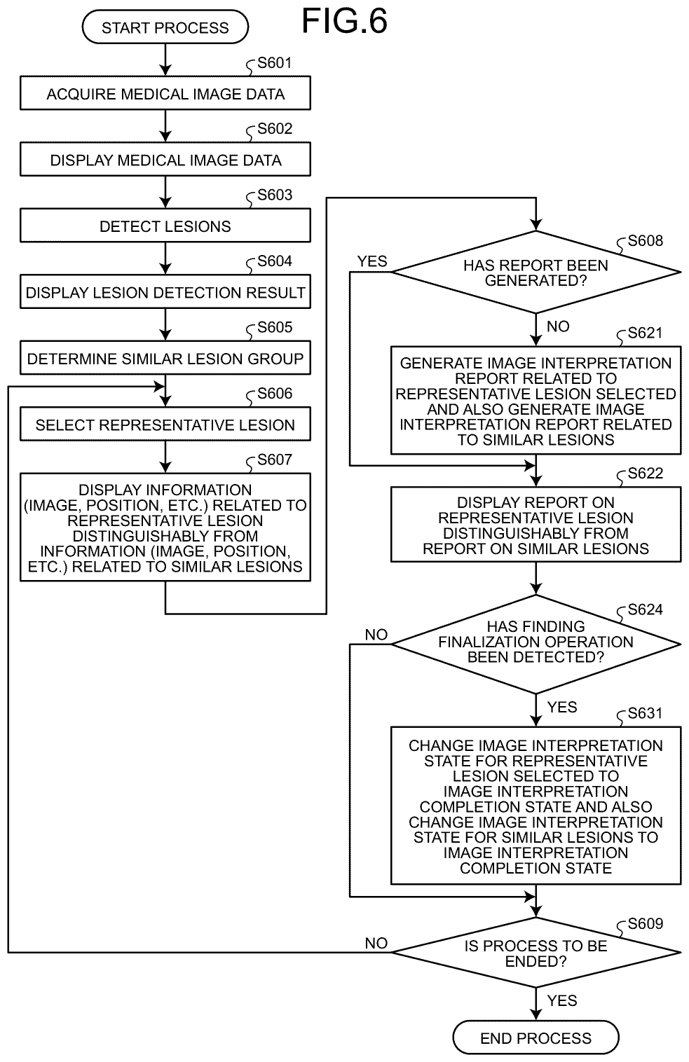

FIG. 6 is a flowchart illustrating an image display process by the information processing apparatus according to the embodiment. This process is started on the basis of an instruction by another system or a user, after a start of the information processing apparatus 101. Upon a start of the process, a medical image to be subjected to image interpretation is specified.

At Step S601, the medical image data acquisition unit 311 acquires the medical image data 321-i (i=1, 2, 3, . . . ) specified at the start, from the case DB 102 via the LAN 103.

At Step S602, the display unit 315 displays the medical image data 321-i (i=1, 2, 3, . . . ) acquired, in the medical image display area 401 on the user interface screen 400.

At Step S603, the lesion acquisition unit 312 detects lesions from the medical image data 321-i (i=1, 2, 3, . . . ) acquired.

At Step S604, on the basis of lesion detection results acquired from the lesion acquisition unit 312, the display unit 315 displays a list of the lesion detection results 421-i (i=1, 2, 3, . . . ).

At Step S605, the group determination unit 313 determines similar lesion groups on the basis of the lesion detection results and the medical image. Furthermore, the display unit 315 displays the numbers of similar lesions 424 in the similar lesion groups determined, in the lesion detection results 421-i (i=1, 2, 3, . . . ). That is, the display unit 315 displays, as information related to the similar lesions forming the same group as a representative lesion, the number of the similar lesions.

At Step S606, the representative lesion selection unit 314 selects, on the basis of detection of specification of a lesion detection result (the lesion detection result 421-1 in the example of FIG. 5) by a user, a lesion corresponding to the specification, as a representative lesion. Furthermore, the display unit 315 highlights the lesion detection result 421-1 corresponding to the representative lesion and also highlights the lesion detection results 421-i (i=2, 4, 6, 8) corresponding to the similar lesions. Furthermore, the display unit 315 displays the representative lesion and the similar lesions distinguishably from each other by displaying a frame of the lesion detection result 421-1 for the representative lesion with a bold solid line and frames of the lesion detection results 421-i (i=2, 4, 6, 8) for the similar lesions with a thin broken line or dotted line. A lesion specified in this embodiment is selected as a representative lesion and is also selected as a representative lesion to be displayed.

At Step S607, the display unit 315 displays information related to the representative lesion and also displays information corresponding to the similar lesions. Furthermore, the display unit 315 displays the representative lesion and the similar lesions distinguishably from each other. The information related to the representative lesion is, for example, the region of interest 511-1, the partial image 521-1, and a rectangle that is part of the lesion position 503. The information related to the similar lesions is the regions of interest 511-2, 511-3, and 511-4, the partial images 521-i (i=2, . . . , 7), and rectangles that are part of the lesion position 503. The information related to the representative lesion and the information related to the similar lesions are displayed such that thicknesses and/or line types of their frame lines are distinguishable from each other. The information related to the lesions and the modes of display are just examples, and the embodiment is not to be limited to these examples.

At Step S608, the report generation unit 316 determines whether or not an image interpretation report related to the representative lesion specified has been generated. In a case where the image interpretation report has been generated (Yes at Step S608), the process advances to processing at Step S622, and in a case where the image interpretation report has not been generated (No at Step S608), the process advances to processing at Step S621.

At Step S621, the report generation unit 316 generates the image interpretation report related to the representative lesion selected and also generates an image interpretation report related to the similar lesions. The image interpretation report is generated by: an inference of image findings from the medical image of the lesion area by use of a CNN that has been trained with training data that are medical images of lesion areas and image findings; and application of the image findings inferred to a template for a report sentence. As to the similar regions, their distribution is evaluated from lesion positions of the similar lesions and a description related to the distribution is generated by use of a template.

At Step S622, the display unit 315 displays the image interpretation report 504-1 related to the representative lesion and the image interpretation report 504-2 related to the similar lesions together. The image interpretation report 504-1 related to the representative lesion and the image interpretation report 504-2 related to the similar lesions are displayed such that thicknesses and/or line types of their underlines are distinguishable from each other.

At Step S624, the image interpretation state management unit 317 determines whether or not finding finalization operation, specifically, click operation on the finding finalization button 541 has been detected. In a case where the finding finalization operation has been detected (Yes at Step S624), the process advances to Step S631. In a case where the finding finalization operation has not been detected (No at Step S624), the process advances to Step S609.

At Step S631, the image interpretation state management unit 317 changes the image interpretation state for the representative lesion selected to the “completed” state and also changes the image interpretation state for the similar lesions to the “completed” state.

At Step S609, a controller not illustrated in the drawings determines whether or not a termination, such as a shutdown of the information processing apparatus 101 or a termination of an application, has been detected. In a case where the termination has been detected (Yes at Step S609), the process is ended, and in a case where the termination has not been detected (No at Step S609), the process from Step S606 is repeated.

As described above, in this embodiment, a similar lesion group is determined on the basis of a predetermined standard from plural lesion candidates acquired, a representative lesion is selected, and information related to similar lesions is displayed together with information related to the representative lesion. Furthermore, the information related to the representative lesion and the information related to the similar lesions are displayed distinguishably from each other. Furthermore, the same kind of processing as processing for the representative lesion is performed also for the similar lesions. Image interpretation is thereby enabled with the representative lesion and the similar lesions being distinguishable from each other, resulting in an effect of enabling efficiency of image interpretation to be increased in a case where multiple lesions have been acquired.

A diagnosis support apparatus described in Patent Literature 1, which an example of a conventional technique, automatically detects features, such as lesion positions and lesion types, and stores the features in a diagnosis image database, and a medical doctor accesses the diagnosis image database and makes a diagnosis by successively displaying images and lesion features. Furthermore, a provisional report generation form including images and data on lesion features is generated and output, and the medical doctor generates a provisional report on the basis of the provisional report generation form.

Furthermore, for example, a medical information processing apparatus and a program described in Patent Literature 2 implement a process of detecting plural types of lesions in a medical image and determining degrees of priority of detected areas of the lesions on the basis of predetermined conditions. Furthermore, display information on the detected areas is generated so that display modes of the detected areas detected are varied according to the degrees of priority determined. A degree of priority is determined on the basis of, for example: whether or not the detected area is a detected area of a lesion of a type specified by a user; whether or not the detected area is present in an area specified by the user; a state, a size, and a position of the detected area; statistical information, such as an occurrence probability of the lesion; and/or a confidence level of the lesion.

The technique described in Patent Literature 1 increases efficiency of report generation for individual lesions detected by generation of provisional reports, but when multiple lesions are detected, reports for the respective lesions need to be finalized. The technique described in Patent Literature 2 enables display of degrees of priority of lesions but a medical doctor needs to check states of the lesions including those having low degrees of priority. That is, when the number of lesions detected increases, work related to image interpretation, such as checking images of the lesions, writing reports, and finalizing the reports, is increased. By contrast, the information processing apparatus 101 according to the embodiment enables efficiency of image interpretation to be increased in a case where multiple lesions have been detected.

### Modified Example of First Embodiment

The representative lesion selection unit 314 may select a representative lesion according to any one or combination of image findings, such as sizes, overall shapes, and margin characteristics of detected lesions. That is, on the basis of any of sizes, shapes, and margin characteristics of lesion candidates acquired by the lesion acquisition unit 312, the representative lesion selection unit 314 may select a representative lesion. Specifically, in a case where the lesion sizes are used, the representative lesion selection unit 314 selects, as a representative lesion, a lesion having the largest lesion size among the similar lesions. In a case where the overall shapes are used, the representative lesion selection unit 314 selects, as a representative lesion, a lesion having a typical overall shape among the similar lesions, that is, an overall shape that a majority of the similar lesions have, and being highest in likelihood that is the probability of the finding that is the overall shape inferred from the medical image of the lesion area. Similarly, in a case where other image findings, such as margin characteristics, are used, the representative lesion selection unit 314 selects, as a representative lesion, a lesion with an image finding that is the same as that for a majority of the similar lesions, the lesion being highest in likelihood that is the probability of the finding that is the image finding inferred from the medical image of the lesion area. For example, the representative lesion selection unit 314 selects, as a representative lesion: a lesion having the highest likelihood of the inference that the lesion is spherical in a case where a majority of the similar lesions are spherical; and a lesion having the highest likelihood of the inference that the lesion is irregular in shape in a case where a majority of the similar lesions are irregular in shape. Similarly, the representative lesion selection unit 314 selects, as a representative lesion: a lesion having the highest likelihood of the inference that the margin is smooth in a case where a majority of the similar lesions have smooth margins; and a lesion having the highest likelihood of the inference that the margin is irregular in a case where a majority of the similar lesions have irregular margins. In a case where a combination of plural selection standards is to be adopted, the representative lesion selection unit 314 selects, as a representative lesion, a lesion having the largest sum total resulting from predetermined weighting of respective items. The lesion sizes are acquired by means of the lesion acquisition unit 312. Furthermore, the image findings, such as the overall shapes and the margin characteristics, are acquired by means of, for example, the CNN that has been described already with respect to the group determination unit 313.

The group determination unit 313 may change the method of determining similar lesions on the basis of classifications of lesions acquired from the lesion acquisition unit 312. That is, the group determination unit 313 may change the method of determining a group on the basis of classifications of lesion candidates. For example, in a case where a lesion is classified as a pulmonary nodule, the group determination unit 313 determines a degree of similarity on the basis of a distance using, for example, an internal concentration, an overall shape, and/or a margin characteristic of the pulmonary nodule. As for hepatophyma, the group determination unit 313 determines a degree of similarity by using, for example, an internal concentration of the phyma, an enhancement pattern over time in dynamic CT, and/or texture inside the phyma. As for encephaloma, the group determination unit 313 determines a degree of similarity by using a concentration around the lesion in a T2-weighted image or FLAIR image, and/or texture in contrast-enhanced T1-weighted image.

The group determination unit 313 may acquire plural features from lesions and select a feature to be used in determination of degrees of similarity on the basis of respective feature distributions. The features herein are, for example, image findings, such as internal concentrations, overall shapes, and margin characteristics of the lesions, or radiomics features. A feature distribution is a histogram having the numbers of lesions plotted for respective features or respective ranges of features. In selecting the feature, the group determination unit 313 selects, for example, a feature having a singular value, specifically, a feature where a lesion isolated from a peak accounting for a majority of the distribution is present in a feature distribution histogram. That is, the group determination unit 313 may acquire plural features in relation to each of lesion candidates, evaluate a distribution for each of the features, select a feature having a singular value on the basis of the distributions, and determine a group on the basis of the feature selected.

Instead of determining lesions having similar lesion forms as a group, the group determination unit 313 may determine a group of lesions on the basis of information other than the forms, such as anatomical positions where the lesions are present, for example, lung segments. Anatomical distances in a lung segment or between lung segments are regarded as degrees of similarity in this case. An anatomical distance is a length as seen in connection with bronchi.

In this embodiment, because representative lesions are selected automatically from similar lesions, at Step S606 in FIG. 6, a representative lesion specified on the basis of user operation from the representative lesions selected automatically is selected as a representative lesion to be displayed.

As described above, in this modified example, a similar lesion group is determined on the basis of a predetermined standard from plural lesion candidates acquired, a representative lesion is selected, and information related to similar lesions is displayed together with information related to the representative lesion. Furthermore, the information related to the representative lesion and the information related to the similar lesions are displayed distinguishably from each other. Furthermore, the same kind of processing as processing for the representative lesion is performed also for the similar lesions. Furthermore, the representative lesion is automatically selected according to a predetermined condition. Furthermore, for the similar lesions, a determination method according to classifications of the lesions is able to be changed automatically. Furthermore, a feature to be used in determination of the similar lesions is able to be selected automatically on the basis of feature distributions. Image interpretation is thereby enabled with the representative lesion and the similar lesions being distinguishable from each other, resulting in an effect of enabling efficiency of image interpretation to be increased in a case where multiple lesions have been acquired. Another effect achieved is that automatic selection of appropriate similar lesions according to classifications of lesions without a user selecting a representative lesion is enabled. Still another effect achieved is that determination of similar lesions by use of histograms of features is enabled, the determination allowing multiple similar lesions and a small number of singular lesions to be distinguished from each other, and determination of similar lesions appropriate for a lesion detected is thus enabled.

### Second Embodiment

An information processing apparatus according to a second embodiment is different from the information processing apparatus according to the first embodiment in that a past image for a medical image to be interpreted is acquired and used in processing. The past image is a medical image having an imaging range that is about the same as that of the medical image to be interpreted and having the same patient captured therein at a time and date prior to that of the medical image to be interpreted. The information processing apparatus according to the second embodiment has a system configuration similar to that described with respect to the first embodiment by use of FIG. 1, a hardware configuration similar to that described with respect to the first embodiment by use of FIG. 2, a functional configuration similar to that described with respect to the first embodiment by use of FIG. 3, a user interface screen similar to that described with respect to the first embodiment by use of FIG. 4 and FIG. 5, and a processing flow similar to that described with respect to the first embodiment by use of FIG. 6, and description thereof will thus be omitted.

The group determination unit 313 according to the second embodiment acquires information related to changes from lesions in the past image corresponding to respective lesions detected, and determines similar lesions on the basis of the information related to the changes (hereinafter, also simply referred to as “changes”). That is, the group determination unit 313 acquires changes from past lesion candidates corresponding to lesion candidates, and determines a group of lesion candidates on the basis of the changes acquired. The information related to the changes is information related to changes in lesion size and/or information related to changes in findings. Specifically, as to the changes in size, the group determination unit 313 determines groups of similar lesions, the groups respectively having differences in predetermined ranges defined beforehand, for example, a range of less than 1 mm, a range of 1 mm or more and 5 mm or less, a range of 5 mm or more and 10 mm or less, and a range of 10 mm or more, the differences being between lesion sizes of the target lesions and lesions sizes of the lesions in the past image. As to the changes in findings, the group determination unit 313 determines lesions as groups having predetermined changes in predetermined findings. These groups of lesions include: a group of lesions having increased spiculations; a group of lesions having increased irregular margins; and a group of lesions that have lost ground glass areas. A medical image selected as the past image has the same patient ID, examined region, and modality as the medical image to be interpreted and has an examination time and date prior to that of the medical image to be interpreted. These patient ID, examined region, and modality are included in the DICOM tag. As to the lesions in the past image, the lesions corresponding to the respective lesions detected, plural landmarks indicating anatomical image features are defined beforehand, and lesions in the past image having positional relations to the landmarks that are about the same as those of the target lesions are selected as the corresponding lesions. The sizes (lesion sizes) of the lesions are acquired by the lesion acquisition unit 312.

The group determination unit 313 may be configured to: determine whether or not any past image is available; determine similar lesions without considering changes in findings over time in a case where any past image is not available; and determine similar lesions in consideration of changes in findings over time in a case where any past image is available. In the case where any past image is available, the group determination unit 313 may increase the weight of a change from a past lesion candidate in determining a group. For example, in the case where any past image is available, the group determination unit 313 may increase the weight to size changes in determining similar lesions. For example, in the case where any past image is not available, the group determination unit 313 determines a group of similar lesions on the basis of distances for internal concentrations, overall shapes, and margin characteristics of lesions, but determines a group of similar lesions by placing larger weight on distances for size changes than other distances in the case where any past image is available. That is, the group determination unit 313 changes the method of determining a group of similar lesions on the basis of whether or not any past image is available.

As described above, in this second embodiment, a similar lesion group is determined on the basis of a predetermined standard from plural lesion candidates acquired, a representative lesion is selected, and information related to similar lesions is displayed together with information related to the representative lesion. Furthermore, the information related to the representative lesion and the information related to the similar lesions are displayed distinguishably from each other. Furthermore, the same kind of processing as processing for the representative lesion is performed also for the similar lesions. Furthermore, lesions having similar size changes from corresponding lesions in a past image are determined as similar lesions. Image interpretation is thereby enabled with the representative lesion and the similar lesions being distinguishable from each other, resulting in an effect of enabling efficiency of image interpretation to be increased in a case where multiple lesions have been acquired. Furthermore, lesions having similar size changes from corresponding lesions in a past image are able to be subjected to image interpretation collectively, resulting in an effect of also enabling efficiency of image interpretation to be increased in follow-up observation of multiple lesions.

### Modified Example of Second Embodiment

In a case where a past image is available, the display unit 315 may acquire changes in size from corresponding lesion candidates in the past image for respective lesion candidates, and display statistical values related to the changes in size per group, or display information related to a lesion candidate having a change in size that is singular in a group. For example, in the case where any past image is available, the display unit 315 acquires size changes from corresponding lesions in the past image respectively for similar lesions, calculates statistical values related to the size changes in the similar lesions, and displays the statistical values. In a case where similar lesions include a lesion having a singular value, the display unit 315 displays information indicating that the lesion has a singular value. Specifically, upon specification of a representative lesion, the display unit 315 displays a size change for the presentative lesion and also displays statistical values of size changes for the similar lesions. The statistical values are, for example, the mean value, the maximum value, the minimum value, the median value, and the standard deviation. Furthermore, a similar lesion having an absolute value equal to or larger than a predetermined value is determined as a similar lesion having a singular value, the absolute value being that of a difference from the mean value, and that similar lesion is displayed distinguishably from the other similar lesions.

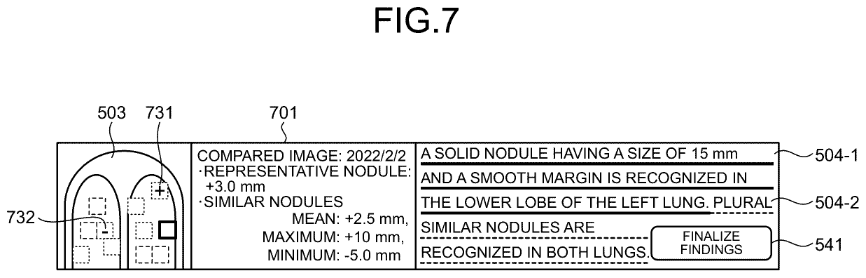

FIG. 7 is a diagram illustrating an example of a user interface screen upon specification of a representative lesion by the information processing apparatus according to the embodiment. Various displays illustrated in FIG. 7 are displayed on the display 207 by the display unit 315 and various kinds of operation by a user are input via the keyboard 209 or the mouse 210.

In FIG. 7, the reference numeral 503 corresponds to the lesion position, the reference numeral 504-1 to the image interpretation report related to the representative lesion, the reference numeral 504-2 to the image interpretation report related to the similar lesions, and the reference numeral 541 to the finding finalization button, which have been described by use of FIG. 5. Furthermore, a reference numeral 701 corresponds to an example of a screen area (hereinafter, referred to as “past comparison information”) where information related to size changes from corresponding lesions in a past image is displayed. Each screen illustrated in FIG. 7 is displayed at a predetermined position on the user interface screen 400.

The past comparison information 701 displayed includes the date of the past image to be compared to, the size change of the representative lesion, and the mean value, the maximum value, and the minimum value of the size changes of the similar lesions. In the example of FIG. 7, the date of the past image is “2022 Feb. 2”, the size change for the representative lesion is “+3 mm”, the mean value of the size changes of the similar lesions is “+2.5 mm”, the maximum value is “+10 mm”, and the minimum value is “−5.0 mm”.

Furthermore, in the lesion position 503, a lesion of the similar lesions including the representative lesion, the lesion with a size change having a singular value, is displayed distinguishably from the other similar lesions. For example, a reference numeral 731 corresponds to a lesion that has increased in size singularly, a reference numeral 732 to a lesion that has decreased in size singularly, and “+” and “−” are respectively displayed additionally to these lesions for these regions to be displayed distinguishably from the other lesions.

As described above, in this modified example, a similar lesion group is determined on the basis of a predetermined standard from plural lesion candidates acquired, a representative lesion is selected, and information related to similar lesions is displayed together with information related to the representative lesion. Furthermore, the information related to the representative lesion and the information related to the similar lesions are displayed distinguishably from each other. Furthermore, the same kind of processing as processing for the representative lesion is performed also for the similar lesions. Furthermore, statistical values of size changes in similar lesions from corresponding lesions in a past image and singular lesions are able to be displayed. Image interpretation is thereby enabled with the representative lesion and the similar lesions being distinguishable from each other, resulting in an effect of enabling efficiency of image interpretation to be increased in a case where multiple lesions have been acquired. Furthermore, lesions that have singularly changed in size from corresponding lesions in a past image are able to be readily distinguished from other lesions, resulting in an effect of also enabling efficiency of image interpretation to be increased in follow-up observation of multiple lesions.

The components of the apparatuses according to the embodiments described above have been functionally and conceptually illustrated in the drawings and are not necessarily configured physically as illustrated in the drawings. That is, specific modes of distribution and integration of each apparatus are not limited to those illustrated in the drawings, and all or part thereof may be configured to be distributed or integrated functionally or physically in any units according to various loads and use situations, for example. Furthermore, all or any part of the processing functions executed in the apparatuses may be implemented by a CPU and a program or programs analyzed and executed by the CPU or implemented as hardware by wired logic.

The information processing methods described above with respect to the embodiments may each be implemented by a computer, such as a personal computer or a workstation, executing a program that has been prepared beforehand. This program may be distributed via a network, such as the Internet. Furthermore, this program may be recorded in a computer-readable non-transitory recording medium, such as a hard disk, a flexible disk (FD), a CD-ROM, an MO, or a DVD, and executed by being read by a computer from the recording medium.

At least one of the embodiments described above enables efficiency of image interpretation to be increased in a case where multiple lesions have been detected.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An information processing apparatus comprising processing circuitry configured to:
acquire plural lesion candidates from a medical image;
determine a group of lesion candidates from the plural lesion candidates acquired;
select a representative lesion candidate from the lesion candidates forming the group; and
display information related to the representative lesion candidate, together with and distinguishably from information related to lesion candidates other than the representative lesion candidate, the lesion candidates forming the same group as the representative lesion candidate.

2. The information processing apparatus according to claim 1, wherein the processing circuitry is configured to acquire degrees of similarity between each of the plural lesion candidates acquired and other ones of the plural lesion candidates acquired and determine the group using the degrees of similarity.

3. The information processing apparatus according to claim 1, wherein the processing circuitry is further configured to perform the same kind of processing for both the representative lesion candidate and the lesion candidates other than the representative lesion candidate, the lesion candidates forming the same group as the representative lesion candidate.

4. The information processing apparatus according to claim 1, wherein the processing circuitry is configured to select the representative lesion candidate in response to reception of user operation.

5. The information processing apparatus according to claim 1, wherein the processing circuitry is configured to select the representative lesion candidate on the basis of any of sizes, shapes, and margin characteristics of the lesion candidates acquired.

6. The information processing apparatus according to claim 2, wherein the processing circuitry is configured to change a mode of display of similar lesions on the basis of the degrees of similarity.

7. The information processing apparatus according to claim 1, wherein the processing circuitry is configured to display, together with a partial image that is an image of part of the medical image, the partial image corresponding to the representative lesion candidate, partial images corresponding to the lesion candidates other than the representative lesion candidate, the lesion candidates forming the same group as the representative lesion candidate.

8. The information processing apparatus according to claim 1, wherein the processing circuitry is configured to display, together with information related to a position of the representative lesion candidate, information related to positions of the lesion candidates other than the representative lesion candidate, the lesion candidates forming the same group as the representative lesion candidate.

9. The information processing apparatus according to claim 3, wherein the processing circuitry is configured to
acquire findings related to the lesion candidates other than the representative lesion candidates, the lesion candidates forming the same group as the representative lesion candidate, according to acquisition of findings for the representative lesion candidate, and
display the findings related to the lesion candidates other than the representative lesion candidates, the lesion candidates forming the same group as the representative lesion candidate, together with the findings for the representative lesion candidate.

10. The information processing apparatus according to claim 3, wherein the processing circuitry is configured to change a state related to image interpretation for the lesion candidates other than the representative lesion candidate, the lesion candidates forming the same group as the representative lesion candidate, according to a change of a state related to image interpretation for the representative lesion candidate.

11. The information processing apparatus according to claim 1, wherein the processing circuitry is configured to
acquire classifications of the lesion candidates in addition to the lesion candidates, and
change a method of determining the group on the basis of the classifications of the lesion candidates.

12. The information processing apparatus according to claim 1, wherein the processing circuitry is configured to acquire plural features in relation to each of the lesion candidates acquired, evaluate distributions for the respective features, select a feature having a singular value on the basis of the distributions, and determine the group on the basis of the feature selected.

13. The information processing apparatus according to claim 1, wherein the processing circuitry is configured to acquire changes from past lesion candidates corresponding to the lesion candidates acquired and determine the group on the basis of the changes.

14. The information processing apparatus according to claim 13, wherein the processing circuitry is configured to increase weight of the changes in determining the group in a case where any past image is available for the medical image.

15. The information processing apparatus according to claim 13, wherein the changes are changes in size of the lesion candidates.

16. The information processing apparatus according to claim 13, wherein the changes are changes in findings for the lesion candidates.

17. The information processing apparatus according to claim 2, wherein the processing circuitry is configured to
display, in order based on the degrees of similarity, the information related to the lesion candidates other than the representative lesion candidate, the lesion candidates forming the same group as the representative lesion candidate, and
determine the group on the basis of a degree of similarity corresponding to a position specified by user operation on the displayed information related to the lesion candidates.

18. The information processing apparatus according to claim 1, wherein the processing circuitry is configured to display, as the information related to the lesion candidates other than the representative lesion candidate and forming the same group as the representative lesion candidate, number of the lesion candidates.

19. The medical information processing apparatus according to claim 7, wherein
the medical image includes images of plural cross sections, and
the processing circuitry is configured to change, in response to operation on the partial images, a position of a cross section to be displayed, the position being in the partial images.

20. The information processing apparatus according to claim 7, wherein in displaying the partial images, the processing circuitry is configured to receive specification of whether to individually enlarge or reduce the partial images on the basis of sizes of the individual lesion candidates acquired, to set the same magnification for the plural lesion candidates, or to set the same magnification as that for display of the medical image.

21. The information processing apparatus according to claim 1, wherein in a case where any past image corresponding to the medical image is available, the processing circuitry is configured to acquire, for each of the plural lesion candidates acquired, a change in size from a corresponding lesion candidate in the past image, and display a statistical value related to these changes per the group or displays information related to a lesion candidate having the change that is singular in the group.

22. An information processing method, including:
acquiring plural lesion candidates from a medical image;
determining a group of lesion candidates from the plural lesion candidates acquired;
selecting a representative lesion candidate from the lesion candidates forming the group; and
displaying information related to the representative lesion candidate, together with and distinguishably from information related to lesion candidates other than the representative lesion candidate, the lesion candidates forming the same group as the representative lesion candidate.

23. A computer-readable non-transitory recording medium having a program recorded therein, the program causing the computer to execute processes of:
acquiring plural lesion candidates from a medical image;
determining a group of lesion candidates from the plural lesion candidates acquired;
selecting a representative lesion candidate from the lesion candidates forming the group; and
displaying information related to the representative lesion candidate, together with and distinguishably from information related to lesion candidates other than the representative lesion candidate, the lesion candidates forming the same group as the representative lesion candidate.
